(19) United States
(12) Patent Application Publication
(10) Pub. No.: US 2019/0376020 A1
(43) Pub. Date: Dec. 12, 2019

(54) OPTICAL CELL CULTURE MONITORING AND ANALYTE MEASURING SYSTEM

(71) Applicant: CORNING INCORPORATED, Corning, NY (US)

(72) Inventors: Scott Robertson Bickham, Corning, NY (US); Ming-Jun Li, Horseheads, NY (US); Gregory Roger Martin, Acton, ME (US); Robert Raymond Raczkowski, Corning, NY (US); Horst Schreiber, Livonia, NY (US); Todd Michael Upton, Eliot, ME (US)

(21) Appl. No.: 16/550,972

(22) Filed: Aug. 26, 2019

Related U.S. Application Data
(63) Continuation of application No. PCT/US2018/049459, filed on Sep. 5, 2018.
(60) Provisional application No. 62/555,338, filed on Sep. 7, 2017.

(51) Int. Cl. C12M 1/00; C12M 1/36; G01N 21/65; C12M 1/26

(52) U.S. Cl. CPC C12M 41/14; C12M 41/48; G01N 2035/00277; G01N 21/65; C12M 33/04; C12M 43/00

(57) ABSTRACT

Methods, systems, and devices for non-invasive measurement of cell cultures are described that provide for remote monitoring of cell status. The system includes a cell culture vessel, at least one monitoring layer, at least one measurement device, and a communication component. The cell culture vessel may include at least one cell culture chamber configured for cell growth and for closed-system operation. The monitoring layer is external to the at least one cell culture chamber. In some cases, the communication component is configured to transmit data from the monitoring layer to a remote location.

### CROSS-REFERENCE TO RELATED APPLICATIONS

This application is a continuation of International Application No. PCT/US2018/049459 filed on Sep. 5, 2018, which claims the benefit of priority to U.S. Provisional Application Ser. No. 62/555,338 filed on Sep. 7, 2017, both applications being incorporated herein by reference.

### BACKGROUND

The following relates generally to cell culture monitoring, and more specifically to a non-invasive monitoring system designed to take measurements in an adherent cell vessel.

Cell cultures are widely used to provide an artificial environment for cell growth. In some cases, a stacked cell culture vessel may provide an increased area for cell growth over single layer dishes. Cells may grow in suspension or attached to a cell culture vessel surface. The processing of cell cultures includes two principal activities, monitoring cell growth and health (confluence and morphology) and ensuring a suitable environment for cell growth (e.g., pH, glucose, and lactate levels). Production cost of cell cultures is extremely high due to low yield, high labor cost, intensive manual work flow, and costly clean room environments where processing is often performed. Monitoring methods of cell cultures are a significant factor for increasing yield and decreasing costs.

Current methods for both viewing cells and measuring analytes can be time consuming and require direct access to the vessel, which risks breaking the sterility of the vessel's environment. Scientists often utilize the naked eye or microscopes to view the confluence of cells. Unfortunately, these methods require direct access to the vessel, which often slows or stops cell growth. Moreover, direct access methods make it difficult or impossible to automate the process. For example, when a stacked cell culture vessel is used, only the exterior layers or near-exterior layers can be monitored, and the status of the interior layers must be estimated without direct measurements.

Cell culture processing is presently monitored (e.g., the presence of certain analytes) through invasive and semi-invasive methods which utilize components such as probe sensors or patches. These methods require some type of contact with the invasive or semi-invasive components within the cell growth environment even though it is desired to allow the systems to operate as a closed system. Monitoring methods are often insufficient and production environments rely on process development techniques for timing the feeding and harvest of cells, which still require manual monitoring.

A closed system with non-invasive monitoring may allow for use of automation to better control cell culture media compositions and cell growth and health. Closed systems can maintain sterility throughout growth, which reduces the requirements and cost of a clean room. Further, real-time monitoring data may be transmitted to a user in a remote location, which can reduce the need of manual labor.

### SUMMARY

The described techniques relate to improved methods, systems, devices, or apparatuses that support non-invasive monitoring of cell cultures. Generally, the described techniques provide for closed-system operation of an adherent cell culture vessel with a monitoring layer external to cell growth layers. The monitoring layer may include a confluence monitor and an analyte monitor. Cell status may be transmitted from the monitoring layer to a user in a remote location. The monitoring layer may be positioned between cell growth layers within a stacked cell culture vessel, and take measurements of the interior layers of the stack. The closed system remains sterile and able to continuously grow cells, for example by remaining in an incubator, while taking real-time cell status data.

A remote monitoring system for non-invasive measurement of a cell culture is described. The system may include a cell culture vessel including at least one cell culture chamber configured for cell growth and for closed-system operation, the at least one cell culture chamber having at least one surface to which cells adhere, at least one monitoring layer including at least one measurement device, wherein the monitoring layer is external to the at least one cell culture chamber, and a communication component configured to transmit data from the monitoring layer to a remote location.

A method for non-invasive measurement of cell cultures is described. The method may include positioning an external monitoring layer between two or more cell culture chambers of a cell culture vessel configured for closed-system operation, the two or more cell culture chambers having at least one surface each to which cells adhere, measuring cell status of the two or more cell culture chambers while allowing continuous cell growth, and transmitting cell status data from the external monitoring layer to a remote location.

In some examples of the system and method described above, the monitoring layer is positioned between two or more cell culture chambers and is configured for inter-layer monitoring. In some cases, the monitoring layer may include at least two measurement devices and may be configured to measure both the cell culture chamber above and below the monitoring layer simultaneously.

In some examples of the system and method described above, the at least one measurement device includes one or both of a confluence monitor or an analyte monitor.

In some examples of the system and method described above, the confluence monitor includes an optical device configured to capture an image of at least one cell culture chamber. In one example, the optical device includes: at least one lens, a mirror, a camera, and the communication component. In another example, the optical device includes: a fiber probes, a mirror, a camera, and the communication component.

In some examples of the system and method described above, the analyte monitor includes a spectral element configured to emit excited light and capture emission light from a media layer within the cell culture chamber. In some cases, the spectral element includes: a light gate, a diffraction grating, a lens, a detector, and the communication component. In some examples of the system and method described above, the spectral element is configured to perform Raman spectroscopy on the media layer.

In some examples of the system and method described above, the monitoring layer comprises polystyrene. In some examples of the system and method described above, the at least one measurement device is removable from the monitoring layer.

In some examples of the system and method described above, the communication component is configured to transmit the data in real time or on demand.

Some examples of the system and method described above may further include processes, features, or means for measuring at least one cell culture chamber above the monitoring layer and at least one cell culture chamber below the monitoring layer simultaneously.

In some examples of the system and method described above, measuring the at least one cell culture chamber above the monitoring layer includes taking a confluence measurement of cells, and measuring the at least one cell culture chamber below the monitoring layer includes taking an analyte measurement of media.

In some examples of the system and method described above, transmitting cell status data is in real-time. In some examples of the system and method described above, transmitting cell status data is on a wireless network.

In some examples of the system and method described above, positioning the external monitoring layer includes positioning one or more measurement devices in the monitoring layer. In some cases, positioning the external monitoring layer further includes positioning the one or more measurement devices at different points on the monitoring layer.

Further scope of the applicability of the described methods and systems will become apparent from the following detailed description, claims, and drawings. The detailed description and specific examples are given by way of illustration only, since various changes and modifications within the spirit and scope of the description will become apparent to those skilled in the art.

### BRIEF DESCRIPTION OF THE DRAWINGS

A further understanding of the nature and advantages of the present disclosure may be realized by reference to the following drawings. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 illustrates a perspective view of an example of a system for non-invasive measurement of a cell culture that supports remote monitoring in accordance with aspects of the present disclosure.

FIG. 2 illustrates a perspective view of an example of a stacked cell culture vessel system for non-invasive measurement of a cell culture that supports remote monitoring in accordance with aspects of the present disclosure.

FIG. 3 illustrates a perspective view of an example of a monitoring layer that supports non-invasive measurement and remote monitoring of a cell culture in accordance with aspects of the present disclosure.

FIG. 4 illustrates a side view of an example of a confluence monitor that supports non-invasive measurement and remote monitoring of a cell culture in accordance with aspects of the present disclosure.

FIG. 5 illustrates a side view of an example of an analyte monitor that supports non-invasive measurement and remote monitoring of a cell culture in accordance with aspects of the present disclosure.

FIG. 6 illustrates a side view of an example of another analyte monitor that supports non-invasive measurement and remote monitoring of a cell culture in accordance with aspects of the present disclosure.

FIG. 7 illustrates a top view of an example of a monitoring layer that supports non-invasive measurement and remote monitoring of a cell culture in accordance with aspects of the present disclosure.

FIG. 8 illustrates a side view of an example of a system for non-invasive measurement of a cell culture that supports remote monitoring in accordance with aspects of the present disclosure.

FIG. 9 illustrates a method for non-invasive measurement of a cell culture that supports remote monitoring in accordance with aspects of the present disclosure.

### DETAILED DESCRIPTION

The singular forms “a,” “an” and “the” include plural referents unless the context clearly dictates otherwise. The endpoints of all ranges reciting the same characteristic are independently combinable and inclusive of the recited endpoint. All references are incorporated herein by reference.

As used herein, “have,” “having,” “include,” “including,” “comprise,” “comprising” or the like are used in their open ended sense, and generally mean “including, but not limited to.”

All scientific and technical terms used herein have meanings commonly used in the art unless otherwise specified. The definitions provided herein are to facilitate understanding of certain terms used frequently herein and are not meant to limit the scope of the present disclosure.

The present disclosure is described below, at first generally, then in detail on the basis of several exemplary embodiments. The features shown in combination with one another in the individual exemplary embodiments do not all have to be realized. In particular, individual features may also be omitted or combined in some other way with other features shown of the same exemplary embodiment or else of other exemplary embodiments.

In practice, cell culture systems that allow for certain measurements to be completed in real time without disturbing the cells, or in other words, a closed system, may facilitate maintaining a sterile cell growth environment. For example, a monitoring system that is external to a cell culture chamber may provide a non-invasive method for measuring cell status, such as cell growth and health, without directly contacting the cells and without contaminating the growth environment. As used herein, the term “closed system” refers to a system wherein the contents of the system are not open to the surrounding atmosphere. The system may include a closure apparatus, such as a cap, which limits or prevents the introduction of contaminants from the surrounding atmosphere. The system may be, but is not necessarily, sealed to ensure sterility of the contents of the system.

As described herein, a cell culture vessel may include a monitoring layer including at least one of optical technology (e.g., micro lens arrays and waveguides) and spectral analytical technology integrated into any of the walls of the cell culture vessel. As will be described in more detail below, according to embodiments of the present disclosure, a monitoring layer including one of optical technology and spectral analytical technology may be integrated into one of the walls of the cell culture vessel, and the other of optical technology and spectral analytical technology may be integrated into another of the walls of the cell culture vessel. The cell culture vessels described herein may be an adherent cell culture vessel generally including a planar surface on which cells adhere while being cultivated.

Alternatively, the monitoring layer as described herein may include a tray positioned external to the cell culture vessel, the tray including at least one of optical technology and spectral analytical technology. As will be described in more detail below, according to embodiments of the present disclosure, a first tray including one of optical technology and spectral analytical technology may be positioned one of above or below the cell culture vessel, and a second tray including the other of optical technology and spectral analytical technology may be positioned at the other of above or below of the cell culture vessel. According to embodiments of the present disclosure, a monitoring layer may be inserted between, but external to, cell growth layers in a stacked vessel utilizing optical technology and spectral analytical technology. This configuration enables the monitoring of cell confluence and measuring analytes with spectral interrogation that illuminates, receives, and processes the signature wavelengths. A communication component may be utilized to transmit monitoring data from the monitor or monitoring layer to a user in a remote location. This configuration may be implemented in single use or multi-use stacked vessels.

Having a monitoring layer positioned external to a cell culture chamber may maintain sterility and enable remote and automated control of the process. By remotely monitoring cell confluence, embodiments of the present disclosure enable the operator to increase the yield in cell processing by optimizing the timing of next steps in cell production, thus reducing handling and reducing operating costs. This disclosure provides a mechanism to automate system controls allowing the operator to be a less skilled technician, lowering labor costs. With the external and remote implementation, the disclosure provides the main component to a closed cell production system that may now be operated in a less costly environment.

The monitoring layer as described herein may be made of polystyrene. This monitoring layer enables two monitoring functions of the cell growth areas: cell confluence and analyte measurement. The confluence monitor may employ a dual lens system with a mirror formed within the monitoring layer, and an attached camera may provide light, image capture, magnification, and image delivery to a user. The analyte monitor may include a spectral analytical technology system and may further include a waveguide system with diffraction grating and lens in the monitoring layer, fibers for excitation and emission may be attached to the monitoring layer and may also be connected to a spectral sensor system.

An exemplary confluence monitor may employ a dual lens system with a mirror for reflecting light at right angles to the cell growth surface for illumination and image capture. The camera may provide the light and image capture function. Light waves or beams may travel through the lens to the mirror where it is focused on an area within the cell growth area. The illuminated image is then received by the camera once passing through the lens.

An exemplary analyte monitor may include a waveguide array that may be located above, below, or in-between stacked cell culture chambers. The monitor may employ dual optical ports where one port may be for excitation light and the other port may be for emission light. The excitation light may travel along the light guide (e.g., waveguide) to the diffraction grating and lens where it reflects off the diffraction grating into the media of a cell culture chamber. The emission fiber may receive the light from the excitation state of the media and deliver the excitation light to the spectral sensor (e.g., detector) to produce an emission or adsorption spectrum. The spectral sensor may include a 2D detector array system. The lens and diffraction grating system may be fabricated in a monitoring layer inserted between the cell culture chambers in stacked adherent cell vessels.

In some examples, micro lenses may be used in combination with the analyte monitor. For example, a micro lens array may be positioned on the top or bottom of the monitoring layer. Micro lens arrays are useful for refracting waves similar to a full-size lens. For example, micro lens arrays may be used for fiber couplings and optical switching. A micro lens may be manufactured in fused silica or silicon by photolithography to produce precise lenses.

Aspects of the disclosure are initially described in the context of a cell culture system. Aspects of the disclosure are further illustrated by and described with reference to apparatus diagrams, system diagrams, and flowcharts that relate to non-invasive remote measurements.

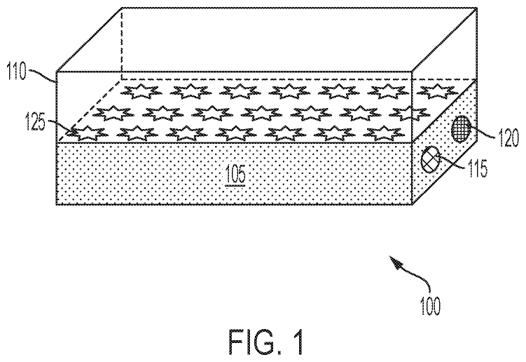

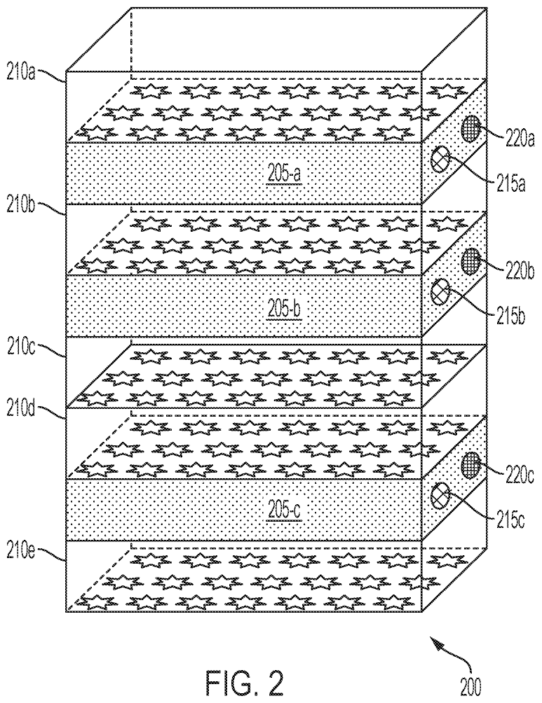

FIG. 1 shows a perspective view of one example of a system 100 for non-invasive measurement of a cell culture chamber 110 that supports remote monitoring in accordance with various aspects of the present disclosure. The non-invasive measurement system 100 includes a monitoring layer 105, a cell culture chamber 110, a confluence monitor 115, an analyte monitor 120, and cells 125. In some aspects, the system 100 may be configured to operate in a wide temperature range, for example the system 100 may operate in an incubator configured for cell growth. In some examples, the non-invasive measurement system 100 may be part of a stacked cell culture vessel as shown in FIG. 2.

In one example, the monitoring layer 105 may be integrated into one or more sides of the cell culture chamber 110. As illustrated, the monitoring layer 105 may be integrated into the side of the cell culture chamber 110 with the surface that the cells adhere to. The one or more sides that include a monitoring layer 105, may be the same thickness or different in thickness than sides that do not include a monitoring layer. The monitoring layer 105 may include at least one of a confluence monitor 115 or an analyte monitor 120. In some aspects, when more than one monitor is present, the monitors may be on the same edge of the monitoring layer 105. In another aspect, the monitors may be on different edges of the monitoring layer 105. When more than one monitor is present, each monitor may be positioned at different heights on the same or different edges of the monitoring layer 105. In some examples, the monitors may be aligned on the edge of monitoring layer 105. As described herein, a monitoring layer 105 below a cell culture chamber 110 may refer to the bottom side of the cell culture chamber 110, a monitoring layer 105 above a cell culture chamber 110 may refer to the top side of the cell culture chamber 110, etc.

In another example, the monitoring layer 105 may be positioned adjacent to the cell culture chamber 110. For example, the monitoring layer 105 may be positioned below the cell culture chamber 110 such that the adherent cells would be on the side of the cell culture chamber 110 adjacent to the monitoring layer 105 as shown in FIG. 1. In another example, the monitoring layer 105 may be positioned above the cell culture chamber 110 such that the adherent cells would be on the side of the cell culture chamber 110 furthest from the monitoring layer 105. The monitoring layer 105 may include at least one of a confluence monitor 115 or an analyte monitor 120. In some aspects, when more than one monitor is present, the monitors may be on the same edge of the monitoring layer 105. In another aspect, the monitors may be on different edges of the monitoring layer 105. When more than one monitor is present, each monitor may be positioned at different heights on the same or different edges of the monitoring layer 105. In some examples, the monitors may be aligned on the edge of monitoring layer 105. The configuration of the confluence monitor 115 and the analyte monitor 120 illustrated in FIG. 1 shows an example of when the two monitors are aligned on the same edge of the monitoring layer 105. In some examples, the monitoring layer 105 may be composed of polystyrene or a similar polymer.

The confluence monitor 115 and the analyte monitor 120 may optically capture the cell status of the cells 125 in the cell culture chamber 110. In some aspects, the confluence monitor 115 and the analyte monitor 120 may include a communication component for transmitting data, such as cell status data, from the monitors to a remote location via a wired communication network or a wireless communication network. For example, the communication component of each monitor may include a Wi-Fi transceiver.

FIG. 2 shows a perspective view of one example of a stacked cell culture vessel system 200 that can be used for non-invasive measurement of a cell culture 210 with support for remote monitoring in accordance with aspects of the present disclosure. The stacked cell culture vessel system 200 may include a plurality of monitoring layers 205, a plurality of cell culture chambers 210, a plurality of confluence monitors 215, and a plurality of analyte monitors 220.

As illustrated, the stacked cell culture vessel system 200 may be configured with a cell culture chamber 210a on top, a monitoring layer 205a below the cell culture chamber 210a and above a cell culture chamber 210b, another monitoring layer 205b below the cell culture chamber 210b and above a cell culture chamber 210c, a cell culture chamber 210d below the cell culture chamber 210c and above a monitoring layer 205c, and a cell culture chamber 210e below the monitoring layer 205c. In some examples, the plurality of monitoring layers 205 are dispersed between the plurality of cell culture chambers 210. The stacked cell culture vessel is not limited to this arrangement. For example, one cell culture chamber 210 (e.g., 210a and 210b) may be positioned on both the top and bottom of a monitoring layer 205 (e.g., 205a) or more than one cell culture chamber 210 may be positioned on both the top and bottom of a monitoring layer 205. The number of cell culture chambers 210 above the monitoring layer 205 may be different from the number of cell culture chambers 210 below the monitoring layer 205 (e.g., 205b and 205c).

Additionally, the stacked cell culture vessel system 200 may be configured to operate over a wide temperature range such as in an incubator at a temperature designed for cell growth.

The confluence monitors 215 and the analyte monitors 220 may capture the cell status of the cells 225 in all cell culture chambers 210, including inter-layer measurements and monitoring. In some cases, the confluence monitors 215 and the analyte monitors 220 may measure the cell status of multiple stacked cell culture chambers 210 with a single monitor. The confluence monitors 215 and the analyte monitors 220 may take measurements of the cell status of the cell culture chambers above and below the monitoring layer 205 that includes the monitors. For example, the confluence monitor 215a may measure the cell growth of the cell culture chamber 210a, the confluence monitor 215b may measure the cell growth of the cell culture chamber 210b, and the confluence monitor 215c may measure the cell growth of the cell culture chambers 210c, 210d, and 210e. In one example, the analyte monitor 220a may measure the cell health of the cell culture chambers 210a and 210b, the analyte monitor 220b may measure the cell health of the cell culture chambers 210c and 210d, and the analyte monitor 220c may measure the cell health of the cell culture chamber 210e.

In some aspects, each confluence monitor 215 and each analyte monitor 220 may include a communication component for transmitting data, such as cell status data, from the monitors to a remote location via a wired communication network or a wireless communication network. For example, the communication component of each monitor may include a Bluetooth transceiver. In other aspects, each monitoring layer 205 may include a communication component for transmitting data, such as cell status data, from the monitoring layer to a remote location via a wired communication network or a wireless communication network. For example, the communication component of each monitoring layer may include a Bluetooth transceiver.

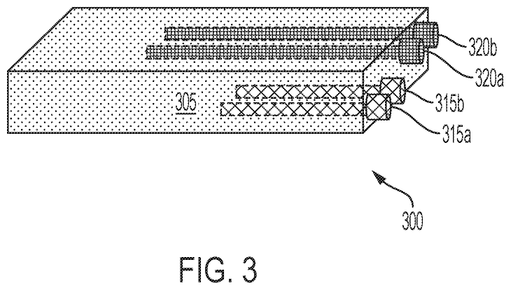

FIG. 3 illustrates an example of a perspective view of a monitoring layer 305 that supports non-invasive measurement and remote monitoring of a cell culture in accordance with aspects of the present disclosure. The monitoring layer 305 may include one or more confluence monitors 315 and one or more analyte monitors 320. In some cases, the monitors 315 and 320 are removable and positionable in different configurations within the monitoring layer 305.

In one example, the monitoring layer 305 may be integrated into one or more sides of a cell culture chamber. In another example, the monitoring layer 305 may be positioned adjacent to a cell culture chamber.

Each confluence monitor 315 and analyte monitor 320 may take measurements of cell status in one direction, such as up or down, once the monitors are positioned in the monitoring layer 305. The confluence monitors 315 and analyte monitors 320 may take measurements in the same direction or in different directions. For example, the confluence monitor 315a and the analyte monitor 320b may monitor the cell culture chamber(s) above the monitoring layer 305, and the confluence monitor 315b and the analyte monitor 320a may monitor the cell culture chamber(s) below the monitoring layer 305. In another example, the confluence monitors 315a, 315b may monitor the cell culture chamber(s) above the monitoring layer 305, and the analyte monitors 320a, 320b may monitor the cell culture chamber(s) below the monitoring layer 305.

When a confluence monitor 315 is integrated into a side of the cell culture chamber, image magnification to monitor cell growth may occur external to the monitoring layer 305. For example, a light pipe may be used within the confluence monitor 315 to transfer the cell surface image to the wall of the monitoring layer 305 without magnification. The image may then be magnified by an external microscope, which may be aimed at the outside edge of the cell culture chamber that includes a confluence monitor 315. In this example, the thickness of the monitoring layer 305 integrated within the side of a cell culture chamber may be decreased.

When an analyte monitor 320 is integrated into a side of the cell culture chamber, light may be transmitted into the cell culture chamber from a light source external to the monitoring layer 305 to monitor cell health by measuring the composition of the media in the cell culture vessel. The light source may extend from the outer edge of the analyte monitor 320 relative to the monitoring layer 305. In this example, the thickness of the monitoring layer 305 integrated within the side of a cell culture chamber may be decreased.

In some aspects, when more than one monitor is present, the monitors may be on the same edge of the monitoring layer 305 as shown. In another aspect, the monitors may be on different edges of the monitoring layer 305. When more than one monitor is present, each monitor may be positioned at different heights on the same or different edges of the monitoring layer 305. In some examples, the monitors may be aligned on the edge of monitoring layer 305. The configuration of the confluence monitors 315 and analyte monitors 320 illustrated in FIG. 3 shows an example of when the monitors are positioned at different heights on the same edge of monitoring layer 305. The analyte monitors 320 are positioned closer to the top of the monitoring layer 305 than the confluence monitors 315. In some cases, the monitoring layer 305 may be composed of polystyrene or a similar polymer. Monitors of the monitor tray 305 are not limited to confluence or analyte monitors and may include other external cell monitors. The shape of monitoring layer 305 may include a rectangular prism or other geometric shapes.

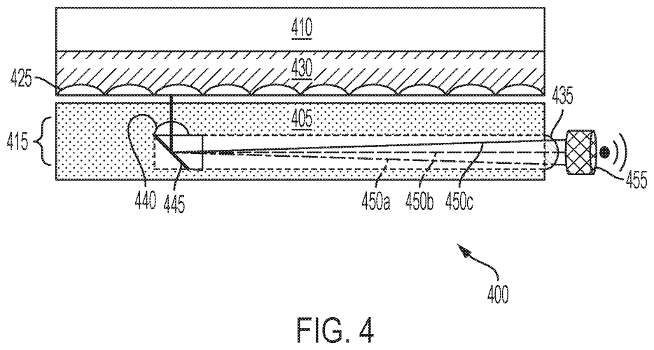

FIG. 4 shows a side view of an example of a confluence monitoring system 400 that supports non-invasive measurement and remote monitoring of a cell culture in accordance with aspects of the present disclosure. The confluence monitoring system 400 may include a monitoring layer 405, a cell culture chamber 410, a confluence monitor 415, cells 425, media 430, lenses 435, 440, mirror 445, and light beams 450. The confluence monitoring system 400 may include one or more monitoring layers 405, one or more cell culture chambers 410 including cells 425 and media 430, and one or more confluence monitors 415 per monitoring layer 405. The monitoring layer 405 may be integrated within a wall of the cell culture chamber 410.

The confluence monitor 415 may take measurements of the cells 425 in the cell culture chamber 410 above the monitoring layer 405 by any optical means. In one example, the confluence monitor 415 uses a 2D imaging array to measure the cell growth above or below the monitoring layer 405. In another example, the confluence monitor 415 may employ a multi-lens (e.g., dual lens 435, 440) system with at least one mirror 445 and camera 455. The confluence monitor 415 may include a sheath or lumen that allows the lens and mirror system to move within the monitoring layer 405 in order to image different sections of the cell culture chamber 410.

Confluence monitor 415 including light beams 450, lenses 435, 440, and mirror 445 may be configured to use a number of illumination options (e.g., reflected light illumination, epi-illumination, dark field illumination, light field illumination, etc.) to observe the cells 425. Light beams 450 may be transmitted from the camera 455 through the first lens 435, where the light beams 450a and 450c may be refracted and focused towards mirror 445. Once the light beams 450 contact the mirror 445, the light beams may be reflected at any angle, for example 90 degrees, to be directed through the second lens 440 into the cell culture chamber 410 to measure the growth of cells 425. The camera 455 may capture the illuminated cells to produce an image of their real-time confluency that may be used to monitor growth over time. As discussed above, the confluence monitor 415 may be designed to image at least one cell culture chamber 410 above or below the monitoring layer 405. The confluence monitor 415 may take measurements of the cell culture chamber 410 above the monitoring layer 405 in order to image the cells on the side with less media 430, which may affect image quality.

The confluence monitor 415 may also include a communication component that transmits cell data to a user, such as the captured image of at least a portion of the cells 425. In some cases, the user may be in a remote location relative to the cell culture, such as a separate room, nearby building, across the world, or on the go. Real-time data related to the cell growth may be transmitted to the user at any remote location. Data transmissions may occur over a wired communication network (e.g., digital subscriber line) or a wireless communications network (e.g., Wi-Fi, Bluetooth, or LTE).

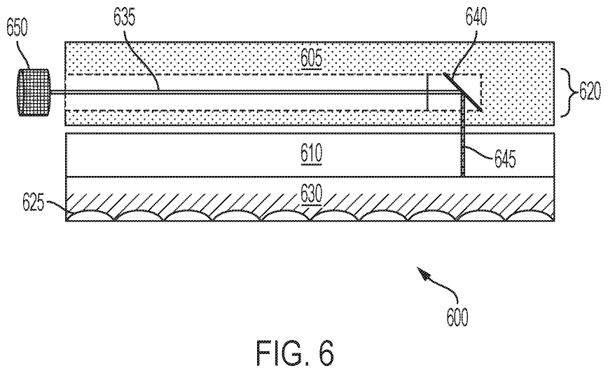

A fiber probe, described in FIG. 6, may be used for confluence monitoring to replace the free space optical system as described in FIG. 4. For example, a multicore fiber or a fiber bundle may be used for transmitting cell images to the camera.

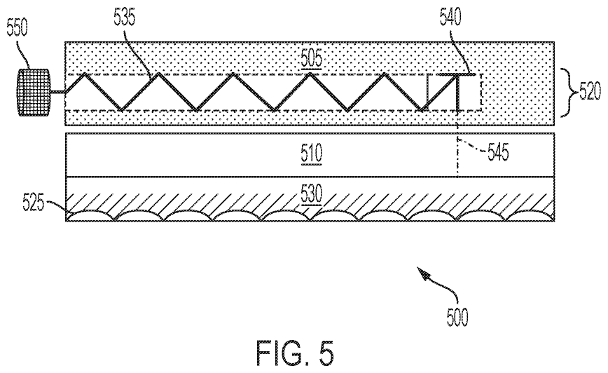

FIG. 5 shows a side view of one example of an analyte monitoring system 500 that supports non-invasive measurement and remote monitoring of a cell culture in accordance with aspects of the present disclosure. The analyte monitoring system 500 may include a monitoring layer 505 and a cell culture chamber 510. The cell culture chamber 510 may include cells 525 and media 530. The analyte monitor 520 may include a sheath or lumen that allows the system to move within the monitoring layer 505 in order to monitor different sections of the cell culture chamber 510. The monitoring layer 505 may be integrated within a wall of the cell culture chamber 510.

In some examples, the monitoring layer 505 may include the analyte monitor 520. The analyte monitor 520 may take measurements of the health of the cells 525 by measuring the composition of the media 530 below the monitoring layer 505 by any spectral means (e.g., Raman spectroscopy). The analyte monitor 520 may include a waveguide 535 (e.g., a light pipe), a diffraction grating and lens 540, light beam 545, and detector 550. The waveguide 535 may direct excited light to the diffraction grating and lens 540, which then directs the light beam 545 into the media 530 within the cell culture chamber 510. Excited light may be produced in a number of ways. Based on the composition of the media 530, distinct emission spectrums will be given off and captured by the detector 550. The detector 550 may transmit the captured emission or adsorption spectrum to a user. The user may use software to determine the composition of the media 530 based on the emission or adsorption spectrums. Some examples of analytes that may be measured by analyte monitor 520 include glucose, lactose, and glutamine.

In one example, a light emitting diode (LED) or laser may be in the analyte monitor 520. The LED or laser may be paired with a photodiode detector within the monitoring layer 505. This example may allow the LED or laser and the photodiode detector to be housed inside the analyte monitor 550 partially or completely within the monitoring layer 505.

As discussed above, the analyte monitor 520 may be designed to monitor at least one cell culture chamber 510 above or below the monitoring layer 505. It is preferable for the analyte monitor 520 to take measurements of the cell culture chamber 510 below the monitoring layer 505 in order to transmit excited light into the media 530, while passing through as few other materials as possible in order to produce a clean emission spectrum.

The analyte monitor 520 may also include a communication component that transmits cell data to a user, such as the captured emission spectrum or adsorption spectrum of at least a portion of the media 530. In some cases, the user may be in a remote location relative to the cell culture, such as a separate room or on the go. Real-time data related to the cell health may be transmitted to the user at any remote location. Data transmissions may occur over a wired communication network (e.g., digital subscriber line) or a wireless communications network (e.g., Wi-Fi, Bluetooth, or LTE).

FIG. 6 shows a side view of another example of an analyte monitoring system 600 that supports non-invasive measurement and remote monitoring of a cell culture in accordance with aspects of the present disclosure. The analyte monitoring system 600 may include a monitoring layer 605 and a cell culture chamber 610. The cell culture chamber 610 may include cells 625 and media 630. The analyte monitor 620 may include a sheath or lumen that allows the system to move within the monitoring layer 605 in order to monitor different sections of the cell culture chamber 610. The monitoring layer 605 may be integrated within a wall of the cell culture chamber 610.

In some examples, the monitoring layer 605 may include the analyte monitor 620. The analyte monitor 620 may take measurements of the health of the cells 625 by measuring the composition of the media 630 in the cell culture chamber 610 below the monitoring layer 605 by any spectral means (e.g., Raman spectroscopy). The analyte monitor 620 may include a fiber probe 635 (e.g., a dual clad fiber two multi-mode fibers (MMFs), or a multicore fiber), a mirror 640, a lens 641, light beam 645, and detector 650. The fiber probe 635 may direct excited light to the mirror 640, which then directs the light beam 645 into the media 630 within the cell culture chamber 610. In some examples, the fiber probe 635 may be bent 90 degrees to direct the light beam 645 through the lens 641 into the media 630 within the cell culture chamber 610 and the mirror 640 may be omitted. In some examples, the lens 641 may be integrated to the fiber end using a fiber lens making process. In some cases, the fiber probe may be straight and extend from the detector 650 to the mirror 640. When the fiber probe 635 is configured with a dual clad structure, the central inner core may be used to transmit light beam 645 to the media 630, and the outer core may be used to capture the Raman-scattered light from the media 630. The central inner core may be a single-mode or a multimode core. When the fiber probe 635 includes two MMFs, one MMF may be for transmitting light beam 645 to the media, and the other MMF may be for capturing the Raman-scattered light from the media 630. When the fiber probe 635 is configured with a multicore fiber, one core (e.g., the core in the center) may be used for transmitting light beam 645 to the media 630, and the other cores may be for capturing the Raman-scattered light from the media 630. In some cases, the mirror 640 may include a diffraction grating. In other cases, the mirror 640 may not include a diffraction grating.

Excited light may be produced in a number of ways. In one example, a light emitting diode (LED) or laser may be in the analyte monitor 620 as described above. Based on the composition of the media 630, distinct emission spectrums are given off and captured by the detector 650. Emissions from the cell media 610 may be directed through the fiber probe 635 to the detector 650. Detector 650 may transmit the captured emission or adsorption spectrum to a user. The user may use software to determine the composition of the media 630 based on the emission or adsorption spectrums. Some examples of analytes that may be measured by the analyte monitor 620 include glucose, lactose, and glutamine.

Fiber probe 635 may include two MMFs used for the input and output of light to and from media 630. The MMFs may have a 90 degree bend near the input/output end. In some examples, one MMF may be for directing light beam 645 into the media 630 while the other MMF may be for capturing Raman-scattered light from the media 630. The distance of the input/output end of the fiber probe 635 from the media 630 may be adjusted for different light beam 645 powers and media 630 illumination areas.

In some examples when the fiber probe 635 is straight, mirror 640 may be omitted. In this example, the input/output end of the fiber probe 635 (e.g., two MMFs) may be polished at a 45 degree angle. Then, the input/output end may be coated with metal to create a mirror on the input/output end of the fiber probe 635.

A fiber probe may also be used for confluence monitoring to replace free space optical system as described in FIG. 4. A multicore fiber or a fiber bundle may be used for transmitting cell images to the camera. In some examples, both the confluence monitoring and the analyte monitoring may be done through fiber probes.

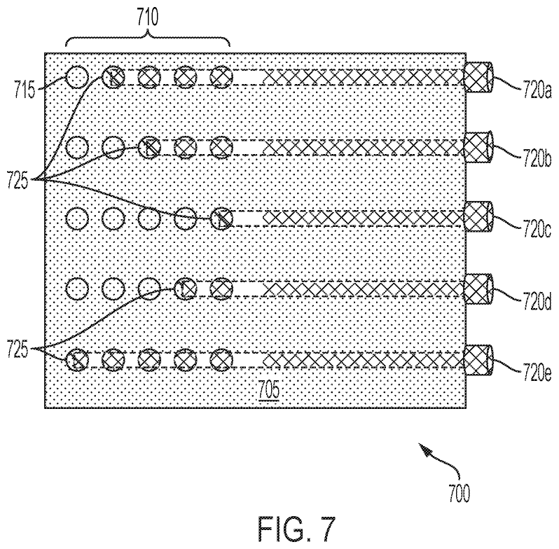

FIG. 7 shows a top view of an example of a monitoring layer system 700 that supports non-invasive measurement and remote monitoring of a cell culture in accordance with aspects of the present disclosure. The monitoring layer system 700 may include a monitoring layer 705 that houses a plurality of analyte monitors 720a-720e. A plurality of lenses 715 may be arranged on the top of the monitoring layer 705 to form a lens array 710 that assists with coupling light into and out of the cell and media samples of a cell culture chamber. In some examples, the lenses may be micro lenses.

The micro lens array 710 may include micro lenses 715 which are not used when the monitoring layer 705 is configured as shown. The micro lens array 710 may also include micro lenses 725 which are used when the monitoring layer 705 is configured as shown. As mentioned above, the analyte monitors 720 may be moved and arranged to monitor different areas of a cell culture chamber. Thus, an array system may be helpful when different points within a cell culture chamber are to be monitored. A micro lens 715 or 725 may be helpful for capturing the emitted light from the media of a cell culture chamber.

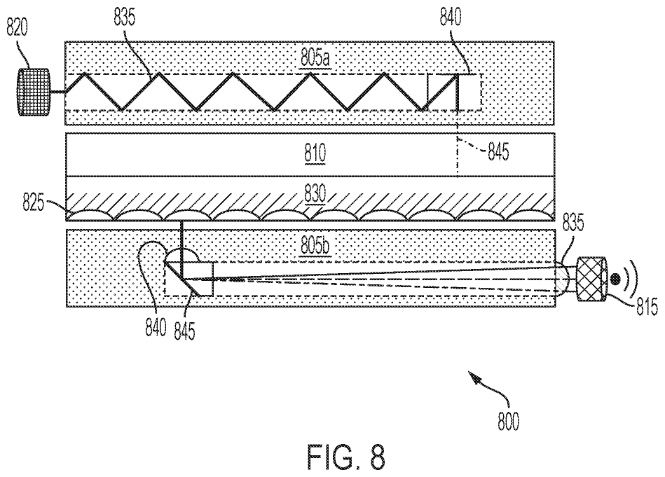

FIG. 8 shows a side view of an example of a system 800 for non-invasive measurement of a cell culture that supports remote monitoring in accordance with aspects of the present disclosure. The system 800 may include two monitoring layers 805a, 805b and a cell culture chamber 810. The monitoring layer 805a may include an analyte monitor 820 that is configured to measure the health of cells 825 by measuring the composition of the media 830 of the cell culture chamber 810 located below the monitoring layer 805a. The monitoring layer 805b may include a confluence monitor 815 that is configured to measure the growth of cells 825 by capturing images of the cells 825 over time.

In some cases, the confluence monitor 815 and the analyte monitor 820 may operate simultaneously or at separate time periods. The confluence monitor 815 and the analyte monitor 820 may also each include a communication component that transmits the captured data in real time to a user. In some examples, the user may be remote.

The system 800 may be configured to operate at a wide temperature range. For example, the system 800 may operate in an incubator at a temperature designed for cell growth. Since the system can operate in an incubator, continuous cell growth with accurate cell culture monitoring in real time is possible. Moreover, real time monitoring may allow for optimized cell growth through improvements in control of the cell culture system, such as an automated feeding schedule.

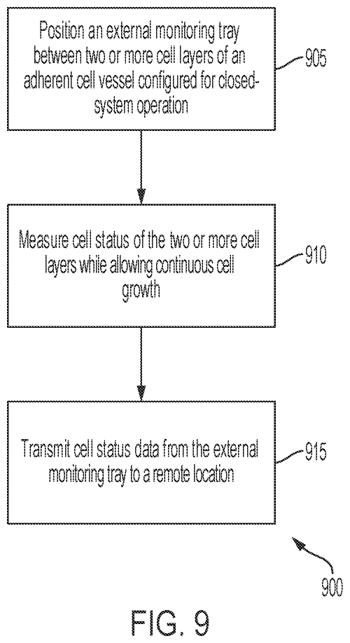

FIG. 9 shows a flowchart illustrating a method for non-invasive measurement of a cell culture that supports remote monitoring in accordance with aspects of the present disclosure. The operations of method 900 may be implemented by any of the systems described above. For example, the operations of method 900 may be performed by systems 100, 200, and 800 as described with reference to FIGS. 1, 2, and 8.

At block 905 the external monitoring layer 205 may be positioned between two or more cell culture chambers 210 of a cell culture vessel configured for closed system operation.

At block 910 the monitoring layer 205 including monitors 215 and 220 may measure the cell status of the two or more cell culture chambers 210 while allowing continuous growth of the cells 225. In certain examples, aspects of the operations of block 910 may be performed by a confluence monitor 215 or an analyte monitor 220 as described with reference to FIGS. 1-8.

At block 915 the one or more communication components may transmit cell status data from the external monitoring layer to a remote location. In certain examples, aspects of the operations of block 915 may be performed by a communication component as described with reference to FIGS. 1-8.

According to an aspect (1) of the present disclosure, a remote monitoring system configured to non-invasively measure a cell culture is provided. The system includes a cell culture vessel comprising at least one cell culture chamber configured to operate as a closed-system, the at least one cell culture chamber having at least one surface to which cells adhere; at least one monitoring layer comprising at least one measurement device, wherein the monitoring layer is external to the at least one cell culture chamber; and a communication component configured to transmit data from the monitoring layer to a remote location.

According to an aspect (2) of the present disclosure, the remote monitoring system of aspect (1) is provided, wherein the monitoring layer is integrated into a wall of the at least one cell culture chamber.

According to an aspect (3) of the present disclosure, the remote monitoring system of aspect (1) is provided, wherein the monitoring layer is positioned between two or more cell culture chambers and is configured for inter-layer monitoring.

According to an aspect (4) of the present disclosure, the remote monitoring system of aspect (3) is provided, wherein the monitoring layer comprises at least two measurement devices and is configured to measure both the cell culture chamber above and below the monitoring layer simultaneously.

According to an aspect (5) of the present disclosure, the remote monitoring system of any of aspects (1)-(4) is provided, wherein the at least one measurement device comprises one or both of a confluence monitor or an analyte monitor.

According to an aspect (6) of the present disclosure, the remote monitoring system of aspect (5) is provided, wherein the confluence monitor comprises an optical device configured to capture an image of at least one cell culture chamber.

According to an aspect (7) of the present disclosure, the remote monitoring system of aspect (6) is provided, wherein the optical device comprises: at least one lens; a mirror; a camera; and the communication component.

According to an aspect (8) of the present disclosure, the remote monitoring system of aspect (6) is provided, wherein the optical device comprises: a fiber probe; a mirror; a camera; and the communication component.

According to an aspect (9) of the present disclosure, the remote monitoring system of aspect (5) is provided, wherein the analyte monitor comprises a spectral element configured to emit one or more excitation wavelengths of light and capture emitted light from a media layer within the cell culture chamber.

According to an aspect (10) of the present disclosure, the remote monitoring system of aspect (9) is provided, wherein the spectral element comprises: a waveguide; a diffraction grating; a lens; a detector; and the communication component.

According to an aspect (11) of the present disclosure, the remote monitoring system of aspect (9) is provided, wherein the spectral element comprises: a fiber probe; a mirror; a detector; and the communication component.

According to an aspect (12) of the present disclosure, the remote monitoring system of aspect (9) is provided, wherein the spectral element is configured to perform Raman spectroscopy on the media layer.

According to an aspect (13) of the present disclosure, the remote monitoring system of any of aspects (1)-(12) is provided, wherein the monitoring layer comprises polystyrene.

According to an aspect (14) of the present disclosure, the remote monitoring system of any of aspects (1)-(13) is provided, wherein the at least one measurement device is removable from the monitoring layer.

According to an aspect (15) of the present disclosure, the remote monitoring system of any of aspects (1)-(14) is provided, wherein the communication component is configured to transmit the data in real time or on demand.

According to an aspect (16) of the present disclosure, a method for non-invasive measurement of cell cultures is provided. The method includes positioning an external monitoring layer between two or more cell culture chambers of a cell culture vessel configured to operate as a closed-system; measuring cell status of the two or more cell culture chambers while allowing continuous cell growth; and transmitting cell status data from the external monitoring layer to a remote location.

According to an aspect (17) of the present disclosure, the method of aspect (16) is provided, wherein measuring the cell status of the two or more cell culture chambers further comprises: measuring at least one cell culture chamber above the monitoring layer and at least one cell culture chamber below the monitoring layer simultaneously.

According to an aspect (18) of the present disclosure, the method of aspect (17) is provided, wherein measuring the at least one cell culture chamber above the monitoring layer comprises taking a confluence measurement of cells.

According to an aspect (19) of the present disclosure, the method of aspect (17) is provided, wherein measuring the at least one cell culture chamber below the monitoring layer comprises taking an analyte measurement of media.

According to an aspect (20) of the present disclosure, the method of any of aspects (16)-(19) is provided, wherein the cell status data is transmitted in real-time.

According to an aspect (21) of the present disclosure, the method of any of aspects (16)-(20) is provided, wherein the cell status data is transmitted over a wireless network.

According to an aspect (22) of the present disclosure, the method of any of aspects (16)-(21) is provided, wherein positioning the external monitoring layer comprises positioning one or more measurement devices in the monitoring layer.

According to an aspect (23) of the present disclosure, the method of aspect (22) is provided, wherein positioning the external monitoring layer further comprises positioning the one or more measurement devices at different points on the monitoring layer.

It should be noted that the methods describe possible implementations, and that the operations and the steps may be rearranged or otherwise modified and that other implementations are possible. Furthermore, aspects from two or more of the methods may be combined.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A cell culture monitoring system adapted to be non-invasively connected to at least one cell culture chamber comprising:
a monitoring layer including at least one cell monitor comprising an optical device configured to capture an image of the at least one cell culture chamber, and at least one analyte monitor comprising a spectral element configured to emit one or more excitation wavelengths of light and capture emitted light from a media layer within the at least one cell culture chamber; and
a communication component configured to transmit data from the monitoring layer to a remote location.

2. The cell culture monitoring system of claim 1, further comprising a cell culture vessel comprising the at least one cell culture chamber configured to operate as a closed-system; and
wherein the monitoring layer is positioned adjacent the cell culture vessel and is configured to non-invasively measure the cell culture vessel.

3. The cell culture monitoring system of claim 2, wherein the monitoring layer comprises at least two cell monitors and at least two analyte monitors and is configured to measure both the cell culture chamber above and below the monitoring layer simultaneously.

4. The cell culture monitoring system of claim 2, wherein the at least one cell culture chamber comprises at least one surface to which the cells adhere.

5. The cell culture monitoring system of claim 1, wherein the monitoring layer is integrated into a wall of the cell culture vessel.

6. The cell culture monitoring system of claim 1, wherein the optical device comprises:
at least one lens;
a mirror; and
a camera.

7. The cell culture monitoring system of claim 1, wherein the optical device comprises:
a fiber probe;
a mirror; and
a camera.

8. The cell culture monitoring system of claim 1, wherein the optical device comprises a sheath or lumen adapted to allow the optical device to image different sections of the cell culture chamber.

9. The cell culture monitoring system of claim 1, wherein the optical device comprises an illumination system adapted to illuminate the cell culture chamber.

10. The cell culture monitoring system of claim 9, wherein the illumination system comprises one or more of a reflected light illumination, epi-illumination, dark field illumination, or light field illumination.

11. The cell culture monitoring system of claim 1, wherein the optical device comprises an illumination system adapted to illuminate cells within the cell culture vessel and means for producing an image of the cells to thereby determine one or more of the confluence, status or morphology of the cells within the cell culture chamber.

12. The cell culture monitoring system of claim 1, wherein the communication component transmits cell culture vessel data to a display component at the location remote from the cell culture vessel.

13. The cell culture monitoring system of claim 1, wherein the spectral element comprises:
a waveguide;
a diffraction grating;
a lens; and
a detector.

14. The cell culture monitoring system of claim 1, wherein the spectral element comprises:
a fiber probe;
a mirror; and
a detector.

15. The cell culture monitoring system of claim 1, wherein the spectral element is configured to perform Raman spectroscopy on the media layer.

16. The cell culture monitoring system of claim 1, wherein the monitoring layer comprises polystyrene.

17. The cell culture monitoring system of claim 1, wherein each of the at least one cell monitor and the at least one analyte monitor is removable from the monitoring layer.

18. The cell culture monitoring system of claim 1, wherein the communication component is configured to transmit the data in real time or on demand.

19. The cell culture monitoring system of claim 1, wherein the monitoring layer is positioned above the at least one cell culture vessel.

20. The cell culture monitoring system of claim 1, wherein the monitoring layer is positioned below the at least one cell culture vessel.

21. A remote cell monitoring system adapted to be non-invasively connected to at least one cell culture chamber comprising:
a monitoring layer adapted to be operatively connected to the at least one cell culture chamber, the monitoring layer having at least one cell monitor including:
an illumination system adapted to illuminate cells within the cell culture chamber;
means for producing an image of the cells to thereby determine one or more of the confluence, status or morphology of the cells within the cell culture chamber; and
a communication component configured to transmit data from the monitoring layer to a remote location.

22. The remote cell monitoring system of claim 21, further comprising a cell culture vessel comprising the at least one cell culture chamber configured to operate as a closed-system; and
wherein the monitoring layer is positioned adjacent the cell culture vessel and is configured to non-invasively measure the cell culture vessel.

23. The cell monitoring system of claim 22, wherein the at least one cell culture chamber comprises at least one surface to which the cells adhere.

24. The remote cell monitoring system of claim 22, wherein the monitoring layer comprises at least two cell monitors and is configured to measure both the cell culture chamber above and below the monitoring layer simultaneously.

25. The remote cell monitoring system of claim 22, wherein the monitoring layer is integrated into a wall of the cell culture vessel.

26. The remote cell monitoring system of claim 21, wherein the means for producing an image comprises:
at least one lens;
a mirror; and
a camera.

27. The remote cell monitoring system of claim 21, wherein the means for producing an image comprises:
a fiber probe;
a mirror; and
a camera.

28. The remote cell monitoring system of claim 21, wherein the monitoring layer comprises polystyrene.

29. The remote cell monitoring system of claim 21, wherein the at least one cell monitor is removable from the monitoring layer.

30. The remote cell monitoring system of claim 21, wherein the communication component is configured to transmit the data in real time or on demand.

31. The remote cell monitoring system of claim 21, wherein the monitoring layer is positioned above the at least one cell culture vessel.

32. The remote cell monitoring system of claim 21, wherein the monitoring layer is positioned below the at least one cell culture vessel.

33. A remote cell monitoring system adapted to be non-invasively connected to at least one cell culture chamber comprising:
a monitoring layer adapted to be operatively connected to the at least one cell culture chamber, the monitoring layer having at least one analyte monitor having a spectral element configured to emit one or more excitation wavelengths of light and capture emitted light from a media layer within the at least one cell culture chamber, the spectral element comprising
a waveguide;
a diffraction grating;
a lens;
a detector; and
a communication component configured to transmit data from the monitoring layer to a remote location.

34. The remote cell monitoring system of claim 33, wherein the spectral element is configured to perform Raman spectroscopy on the media layer.

35. The remote cell monitoring system of claim 33, wherein the monitoring layer comprises polystyrene.

36. The remote cell monitoring system of claim 33, wherein the at least one analyte monitor is removable from the monitoring layer.

37. The remote cell monitoring system of claim 33, wherein the communication component is configured to transmit the data in real time or on demand.

38. The remote cell monitoring system of claim 33, wherein the monitoring layer is positioned above the at least one cell culture vessel.

39. The remote cell monitoring system of claim 33, wherein the monitoring layer is positioned below the at least one cell culture vessel.
